(19) United States
(12) Patent Application Publication
(10) Pub. No.: US 2014/0005531 A1
(43) Pub. Date: Jan. 2, 2014

(54) ORTHOPAEDIC NAVIGATION SYSTEM

(71) Applicant: Nicola Joanne Taylor, East Lavant (GB)

(72) Inventor: Nicola Joanne Taylor, East Lavant (GB)

(21) Appl. No.: 13/995,854

(22) PCT Filed: Dec. 16, 2011
(86) PCT No.: PCT/EP11/73131

§ 371 Date: Sep. 9, 2013

(30) Foreign Application Priority Data
Dec. 20, 2010 (GB) 102675.2

(51) Int. Cl. A61B 5/06; A61B 5/00

(52) U.S. Cl. CPC A61B 5/06; A61B 5/742
USPC 600/424

(57) ABSTRACT

An orthopaedic navigation system (1) has (a) a reference point sensor (10) for connection to a body to transmit a reference position signal; (b) a trial sensor (20) for connection to a trial implant and to transmit a trial position signal indicating its position relative to the reference point sensor; (c) an implant sensor (41) for connection to a first implant and to transmit a first implant position signal indicating its position relative to the reference position sensor; (d) a computer (30) having a receiver to (i) receive said reference point signal and trial position signal to create a predetermined position value, (ii) receive said reference point signal and first implant position signal to create a comparison position value, and having a transmitter to transmit output signals of the predetermined position value; and (e) a screen (51) to receive said visual signals and create a display to compare the predetermined position value relative to the comparison value, which display can be seen by a user while simultaneously viewing the body.

The present invention relates to an orthopaedic navigation system.

When repairing damaged hips it is common practice to use artificial hip bearings. Such bearings often have an acetabular cup component placed in the pelvis which receives a femoral head component. The correct inclination/abduction angle of the cup component is very important to achieve good leg mobility after the operation. Also the correct inclination/abduction angle will vary depending on the type of hip bearing being used. Incorrect positioning increases the likelihood of excessive and damaging wear.

It is known from US 2009/0099665, and a product sold by Finsbury Orthopaedics Ltd, to use lasers as a navigation system to achieve the correct inclination/abduction angle. This product uses a first laser secured to the pelvis. A second laser is secured in a predetermined position to a trial acetabular component. When a surgeon aligns the trial acetabular component to the correct angle relative to the head, the two lasers are turned on and shone at a wall. The first laser is then adjusted until its beam converges with the beam of the second laser on the wall. The surgeon then removes the trial acetabular component and inserts the final acetabular cup component with a cup introducer. The cup introducer has a third laser in the same predetermined position relative to the final acetabular cup component as the second laser on the trial acetabular component.

The surgeon introduces the final acetabular cup component whilst ensuring that the third and first laser beams converge, whereby the final acetabular cup component is at the correct angle.

A problem with this system is that the surgeon has to look at the wall behind to observe the laser beams and make sure they converge whilst performing the operation in front. This can be awkward and many surgeons find this approach difficult.

The invention seeks to provide an alternative, preferably an improved system.

An aim of embodiments of the present invention is to provide a navigation system that will provide positional information for a prosthesis, e.g. a hip implant, enabling a user to obtain information as to accurate positioning of a prosthesis, e.g. so a surgeon can then position and fit an prosthesis, e.g. an acetabular cup, so as to give a optimum range of movement and optimum wear characteristics. In specific embodiments, the present invention provides a navigation system that can be easy to use, compact (fitting directly to the patient or instrumentation used in the operation) and giving the added benefit of supplying alignment information direct to the user

Accordingly, the invention provides an orthopaedic navigation system, for providing information as to the position of a prosthesis compared to a desired position of the prosthesis, comprising:
(a) a first sensor for connection to a prosthesis and to transmit a prosthesis position signal;
(b) a computer having a receiver to receive said prosthesis position signal and create a first position value and programmed to compare said first position value with a second position value corresponding to a position signal from a desired prosthesis position; and
(c) an output to provide information indicating the proximity of the first position value to the second position value.

In use, an operator selects a prosthesis, say an acetabular cup for a hip, and attaches the sensor to the cup or to an instrument for fitting of the cup to the body.

Position information from the sensor is compared with information in the computer relating to the desired position for the cup, typically the position of the tool suitable for correctly fitting the cup and the output provides information indicating the difference between the first position value and the second position value, thereby providing information to the user to indicate whether the tool and/or cup is in the correct position to be fitted. It can then be fitted.

The second position value, also referred to as a target position value, may be obtained by calculation, based for example on data obtained by a CT scan, MRI or ultrasound. This can be stored in the computer for reference by the user. The data may be from previous procedures on one or more other bodies or may be from the body on which the procedure is now being carried out.

The first position value may be obtained with reference to the body. Thus, a second sensor can be provided for connection to a body to transmit a reference position signal. Suitably, the computer is programmed to receive said reference position signal and said prosthesis position signal to create therefrom the first position value. Hence the first position value can be calculated from inputs combining the location of a predetermined part of the body and data from a sensor on an implant.

A third sensor may be used, for connection to a trial prosthesis and to transmit a trial prosthesis signal. For example, in a trial reduction, a user associates the third sensor with a trial implant and moves the trial implant until a suitable position is identified. This will generally be or correspond to the correct, desired position of an actual implant or a tool to fit the actual implant. With the trial implant correctly located, the trial position signal can be transmitted to the computer. The computer is suitably programmed to receive said trial prosthesis signal and create the second position value therefrom.

The computer may in addition be programmed to receive said reference position signal and said trial prosthesis signal and create the second position value therefrom. This enables calculation of the second position value based on two inputs, those from the body and from the trial implant.

The system suitably enables a user to know whether the prosthesis is in or near to the correct position. Typically, the output varies according to the difference between the first position value and the second position value. A change in output can then be used as the user changes the position of the prosthesis to find the correct, target position.

The output can comprise an audible signal or a visual signal. For an audible signal, intermittent tones can be used, for example wherein the tone and the interval between tones varies according to the proximity of the first position value to the second position value. The user can then hear whether in moving the prosthesis it is getting closer to or further away from the target position.

For a visual signal, a display unit can conveniently be provided to display the visual signal. A screen within easy access for the user is suitable. A preferred display is a direct to eye display, especially one that can be used by the person operating the system without having to look up from the body and the prosthesis. A particular advantage is that using the system of the invention, the prosthesis can be moved and the user can see on the direct to eye display whether the prosthesis is correctly aligned in real time and without having to avert the user's eyes from the procedure.

In a particular embodiment of the invention, a navigation system comprises:
(a) a reference point sensor (the second sensor) for connection to a fixed point on a body to transmit a reference position signal;
(b) a trial sensor (the third sensor) for connection to a trial implant and to transmit a trial position signal indicating the position of the trial implant relative to the reference point sensor;
(c) an implant sensor (the first sensor) for connection to a first implant and to transmit a first implant position signal indicating the position of the first implant relative to the reference position sensor;
(d) a computer having a receiver to (i) receive said reference point signal and trial position signal to create a predetermined position value, (ii) receive said reference point signal and first implant position signal to create a comparison position value, said computer having a transmitter to transmit output signals of the predetermined position value; and
(e) an output unit to receive said output signals and to create an output to compare the predetermined position value with the comparison position value.

Very suitably, a screen is included with the system, adapted to receive said output signals, being visual signals and to create a display to compare the predetermined position value relative to the comparison value. The screen is preferably adapted to be seen by an operator whilst simultaneously viewing a patient.

In a further specific embodiment, described in more detail below, there is provided an orthopaedic navigation system for aligning first and second co-operating implants comprising:

a) a reference point sensor for connection to a fixed point on a body to transmit a reference point position signal,
b) a trial sensor for connection to a trial first implant and to transmit a trial position signal indicating the position of the trial implant relative to the reference point sensor,
c) an implant sensor for connection to a first implant and to transmit a first implant position signal indicating the position of the first implant relative to the reference point sensor,
d) a computer having a receiver to i) receive said reference point signal and trial position signal to create a predetermined position value, ii) receive said reference point signal and first implant position signal to create comparison position value, said computer having a transmitter to transmit visual signals of the predetermined position value and comparison position value, and
e) a screen adapted to receive said visual signals and to create a display to compare the predetermined position value relative to the comparison position value, said screen being adapted to be seen by an operator whilst simultaneously viewing a patient.

Also provided by the invention are related kits. A kit of the invention for use in providing information as to the position of a prosthesis compared to a desired position of the prosthesis hence comprises:
(a) a prosthesis comprising a first sensor to transmit a prosthesis position signal;
(b) a computer having a receiver to receive said prosthesis position signal and create a first position value and programmed to compare said first position value with a second position value corresponding to a position signal from a desired prosthesis position; and
(c) an output to provide information indicating the proximity of the first position value to the second position value.

The kit may also contain a second sensor for connection to a body to transmit a reference position signal. The computer may be programmed to receive said reference position signal and said prosthesis position signal to create therefrom the first position value.

An optional additional kit component is a trial prosthesis having a third sensor to transmit a trial prosthesis signal. To use this, the computer may be programmed to receive said trial prosthesis signal and create the second position value therefrom or to receive said reference position signal and said trial prosthesis signal and create the second position value therefrom.

The outputs for the kit are as for the system of the invention. Preferred kits also include a direct to eye display to show how the actual prosthesis position compares to its target position.

A particular kit of the invention comprises:
(a) a reference point sensor for connection to a fixed point on a body to transmit a reference position signal;
(b) a trial implant comprising a trial sensor to transmit a trial position signal indicating the position of the trial implant relative to the reference point sensor;
(c) a first implant comprising an implant sensor to transmit a first implant position signal indicating the position of the first implant relative to the reference position sensor;
(d) a computer having a receiver to (i) receive said reference point signal and trial position signal to create a predetermined position value, (ii) receive said reference point signal and first implant position signal to create a comparison position value, said computer having a transmitter to transmit output signals of the predetermined position value; and
(e) a screen adapted to receive said visual signals and to create a display to compare the predetermined position value relative to the comparison value, said screen being adapted to be seen by an operator whilst simultaneously viewing a patient.

Still further, the invention provides methods of providing information as to the position of a prosthesis compared to a desired position of the prosthesis. Methods of the invention comprise:
(a) associating a first sensor with the prosthesis, said first sensor being capable of transmitting a prosthesis position signal;
(b) operating a computer having a receiver to receive said prosthesis position signal and create a first position value and programmed to compare said first position value with a second position value corresponding to a position signal from a desired prosthesis position; and
(c) generating an output from the computer that indicates the proximity of the first position value to the second position value.

In some methods carried out on a body, the second position value is calculated without direct reference to the body but based for example on data obtained by a CT scan, MRI or ultrasound. The value can then be stored in the computer.

The method may comprise attaching a second sensor to a body to transmit a reference position signal, and the computer may be programmed to receive said reference position signal and said prosthesis position signal and calculate therefrom the first position value.

In some methods, the second position value is obtained during the procedure, optionally with direct reference to the body. One such method comprises associating a third sensor with a trial prosthesis to transmit a trial prosthesis signal, wherein the computer is programmed to receive said trial prosthesis signal and create the second position value therefrom; the computer may also be programmed to receive said reference position signal and said trial prosthesis signal and create the second position value therefrom. In typical use, the method has a step of moving the trial prosthesis until it is in a desired position and then transmitting the trial prosthesis signal. This records a correct or target position for when the actual implant is used in a subsequent step of the method, for example when a user moves the actual prosthesis and notes the output to determine if it is in the correct or target position.

The methods suitably comprise generating an output that varies according to the proximity of the first position value to the second position value. As for the above-described systems and kits, the signal can be audible or visual, and other preferred and optional features of the method correspond to preferred and optional features of the systems and kits. Thus, preferably, the methods comprise using a direct to eye display.

A method of a particular embodiment comprises:
(a) connecting a reference point sensor (the second sensor) to a body to transmit a reference position signal;
(b) connecting a trial sensor (the third sensor) to a trial implant, or associating a trial sensor with a trial implant, to transmit a trial position signal indicating the position of the trial implant relative to the reference point sensor;
(c) connecting an implant sensor (the first sensor) to a first implant, or associating an implant sensor with a first implant, to transmit a first implant position signal indicating the position of the first implant relative to the reference position sensor;
(d) operating a computer having a receiver to (i) receive said reference point signal and trial position signal to create a predetermined position value, (ii) receive said reference point signal and first implant position signal to create a comparison position value, said computer having a transmitter to transmit output signals of the predetermined position value; and
(e) generating an output to compare the predetermined position value with the comparison position value.

The output can be on a screen adapted to receive said visual signals and to create a display to compare the predetermined position value relative to the comparison value, said screen being adapted to be seen by an operator whilst simultaneously viewing a patient. A head mounted display is particularly useful. The output can be on a head mounted display on a user operating the process, optionally on goggles or a helmet to be worn by the user. Also instead of glasses, the screen could be provided on a helmet or a bracket connecting a screen to a head.

The systems, methods and kits are suitable for various prostheses, for example wherein the prosthesis is a hip implant, a knee implant, a shoulder implant or an elbow implant. In kits, the trial prosthesis cam be a corresponding trial implant for performing a trial reduction to determine suitable position for the hip implant, knee implant, shoulder implant or elbow implant

Preferably the position signals are transmitted wirelessly between the computer and the output, e.g. to a direct to eye display or head-up display unit.

Preferably the trial position signal and the first implant or prosthesis position signal position indicate the distance and angle between the reference point and the trial implant and first implant respectively.

Preferably the sensors are orientation/position sensors of the type sold by Fraunhofer or as described in US 2007/0287911, referred to as electronic orientation units (EOUs).

In an embodiment of the invention, a plurality of EOUs are built into surgical instrumentation which in use give their positions in free space. The information from the EOUs can be provided to the user, e.g. projected in front of the user's eye so that the user is able identify the correct position for a prosthesis. This system is both easy to use and mobile, allowing for the system to be moved between locations.

A particular kit of the invention comprises two or three independent EOUs that can be fitted both to the patient and to instrumentation to be used during a procedure. The EOU units in use give their coordinates within free space and by attaining their inclination and relationship to each other using a series of the devices it is possible to use the information to align a prosthesis in the desired position.

A direct to eye display unit or a head-up display unit (HDU) is an optional kit component, It may project an alignment grid in front of the user so that the user can align the prosthesis during the procedure thus allowing the user to focus on the procedure at the same time, without looking away from the patient.

A control unit may be provided that processes the data from the EOUs and transmits this to the HDU or direct to eye display. The control unit may also have a touch screen display so that a visual record of the actual angles from the EOUs can be seen. It is further optional that the control unit stores information relating to different types of prosthesis, e.g. hips, knees, shoulders and elbows, to be fitted, so the user may select the application for the procedure about to be performed. Preferably the control unit is a portable computer, a mobile computer or a smartphone. Preferably the information relating to different types of prosthesis, e.g. hips, knees, shoulders and elbows, to be fitted is processed by using prosthetic-, operation- or patient-specific applications stored in the control unit.

The apparatus and method described herein may be used for alignment of hips and other prosthetic implants such as the knee, shoulder and elbow. The method and apparatus may also used for the alignment of prosthetic limbs and more specifically lower limbs, which need to be correctly adjusted so the amputee has full mobility once they have left hospital. Correct positioning increases the life expectancy of the prosthesis and requires correction less often.

For devices intended to be used for impaction procedures, it is preferred that the EOUs can resist a shock loading of 500 N or greater generated during the impaction procedure. Separately, the EOUs preferably fit into an enclosure that does not exceed 55×37×10 mm. Also separately, it is preferred that all of the electrical enclosures are sterilisable. This may be achieved by provision of a disposable outer case for the units.

In use of the system of the invention, there are many ways to gain three-dimensional (3D) information about the body in a way that can be utilized to provide the second position value and aid position of a prosthesis. These include (i) volumetric images achieved by CT scans, MRI or ultrasound, (ii) fluoroscopic images obtained by attachment of a dynamic reference frame to the bones and a fluoroscope and (iii) imageless navigation collecting kinetic information about joints and morphological information about the bones during surgery. Imageless navigation takes place during surgery, while fluoroscopic and volumetric images can be collected and used pre- or intra-operatively.

In an example of the invention in use, to fit an acetabular cup for a hip prosthesis a trial alignment of the cup position during a trial reduction is carried out (where the user fits the now modified femur into the acetabulum) and the invention is used to reproduce that position with the actual acetabulum cup during impaction. EOUs are positioned on the pelvis and on the instrumentation used for the trial reduction. A trial acetabular cup, with a handle protruding at a fixed angle from the face of the cup, is placed in the acetabulum. At the end of this handle is another EOU sensor. The leg is moved through the desired full range of motion (ROM). The anteversion and inclination of the trial cup is adjusted until a position is found where flexion/extension ROM is possible without impingement and satisfactory abductionadduction is achieved with stability. Once this target position is found, the control unit records the angular relationship between the fixed EOU and instrument handle EOU. In a second stage of the procedure the alignment instrument is removed and the introducer (impaction handle) is introduced. The introducer also has an EOU attached to the handle. The acetabulum cup to be implanted is placed in the acetabulum and the introducer adjusted by the user with the aid of a HDU or direct to eye display unit indicating in real time whether the introducer is in the right position (corresponding to the target position) whilst adjusting the introducer orientation. This series of procedures ensures that the orientation of the cup to be implanted is in the same alignment as determined during the trial reduction. The user can keep the alignment image in the correct position during impact of the cup into the acetabulum socket to correctly locate the cup based on the positional information acquired during the trial reduction.

Two EOUs can be fixed at different positions on the hip to detect the current orientation of the pelvis. The EOU on the working tool detects the orientation of the cup during the operation. The navigation is based on triangulation relationship between orientation measuring devices. The EOU may incorporate three accelerometers, three magnetometers and three gyroscopes and measure the angular orientation on the X, Y & Z axes. The resolution of commercially available sensors for the EOUs can be improved with additional calibration after mounting in a system in cases where the resolution has to be better than 1°. The acceleration and gyroscope sensors generally should be calibrated in bias, scaling/sensitivity and orthogonality; the magnetometers need calibration for bias and orthogonality. The accelerometers detect magnitude and direction of the acceleration as a vector quantity. The gyroscopes use a vibrating element for detection of positional orientation. The gyroscopes improve the recognition of movement within a 3D space when combined with an accelerometer. The drift errors are detected and compensated for by controlling software in the control unit.

The sensor data may be transferred wirelessly to the control unit and analyzed by the data filter.

The filter is based on a set of adaptive state estimators in which the dynamics of the system are modelled and stored. The filter recognises errors in the incoming stream of sensor data and performs a plausibility check on the sensor measurements. Signals which are detected to be valid are accepted and those signals which are faulty are corrected by the filter and subsequently fed into the algorithm. The main task of the sensors and the filter algorithm is the robust and accurate orientation measurement in static and dynamic conditions of the EOUs, alignment tool and patient. The computer or control unit may have a touch screen display based on the capacitive principle with a hard front (similar to smart phones) which are easier to disinfect. This acts as the interface between the EOUs and the user, through which the user is able to select the type of operation from the memory of the control unit, stored in the form of applications so that they can be updated and have other applications added at a later date for different types of operation. The control unit is also the data logger for the documentation of the operation.

Visual information may be provided via a HDU or direct to eye display unit which displays the position of the impaction EOU in front of the user's eye. The screen may be transparent. The screen may be suitable to be in the user's peripheral vision. The image displayed is designed to assist in providing easy to read information about the position of an actual prosthesis to be fitted compared with desired positional information obtained from calculation or from a trial reduction. The image displayed may be in the form of cross hairs which when lined up with second cross hairs will mean that the user has matched the actual prosthesis position with the desired position, i.e. the prosthesis is ready to be deployed, inserted or fitted.

In suitable embodiments, components of the system are mobile and work over a range of maximum 2-5 m. The data can be transferred wireless using one of the Industrial-Science-Medical (ISM) Frequency Bands at 2.4 GHz or 868 MHz (EU only, 915 MHz in US) with short range devices (SRD) working with IEEE 802.15.4 medium access control (MAC) software stack in a wireless body area network (WBAN). The wireless modules on the market are configurable to optimise the transfer conditions (choice of transmitting channel, sending power level and data rate). The wireless modules are small in size and efficient enough for short distances using small ceramic antennas. The standard IEC 60601-1-281 defines a safety distance to other medical devices depending on transmitted power and frequency (e.g. −10 dBm sending power at 2.4 GHz, needing a safety distance of 2.3 cm). The communication in the WBAN may have a point-to-multipoint structure. The WBAN may be mastered by the control unit, which may be the network coordinator, asking the sensor data from the EOUs and transmitting the display data to the HDU or direct to eye display unit. A reason to use IEEE 802.15.4 is the low transferred data rate and low power consumption of the wireless components. The wireless solution preferably fulfils the standards IEC 60601-1-2 and IEC 8000183.

A specific embodiment of the invention will now be described with reference to the accompanying drawings in which:

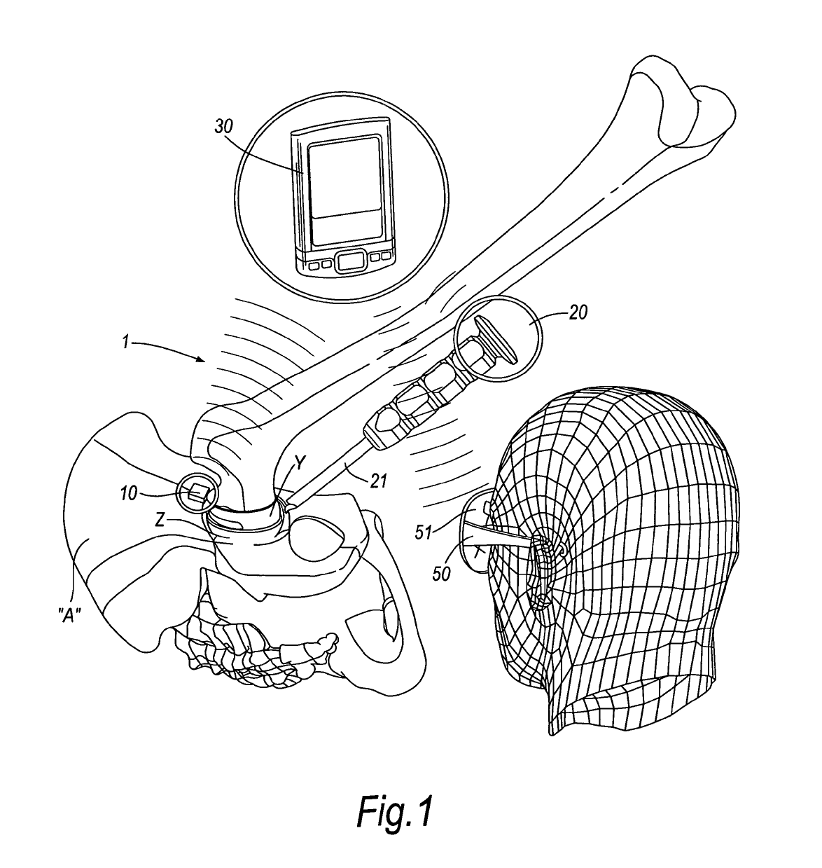

FIG. 1 shows a reference point sensor and trial sensor communicating with a computer and screen, and

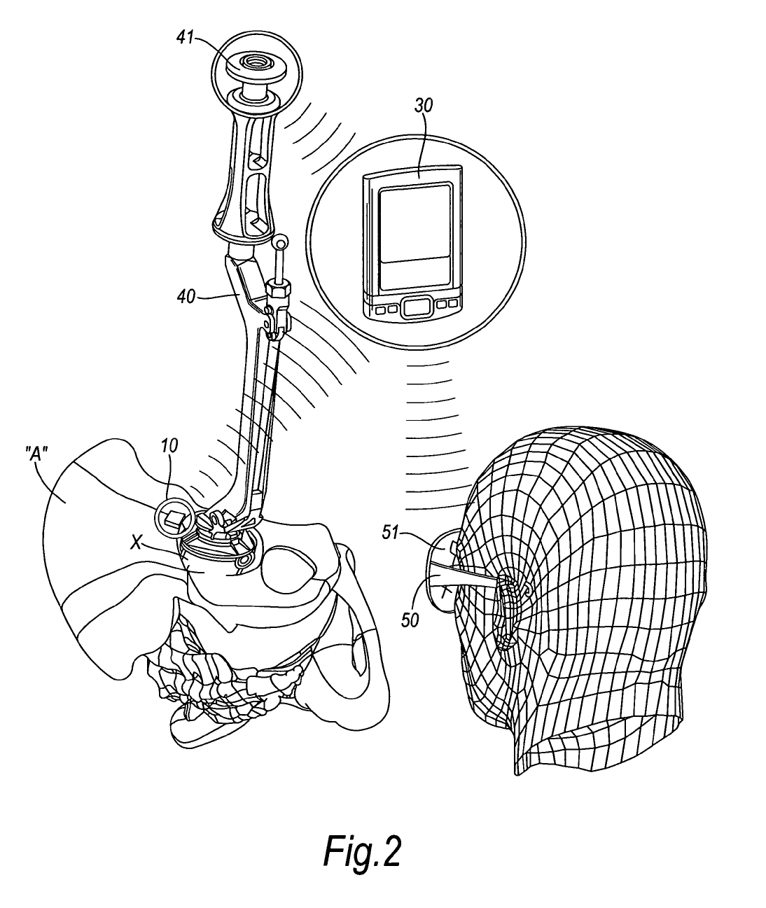

FIG. 2 shows a reference point sensor and implant sensor communicating with a computer and screen.

Referring to the drawings there is shown an orthopaedic navigation system 1 for aligning first and second co-operating hip replacement implants in the form of acetabular cup X component placed in the pelvis which receives a femoral head component Y.

A reference point sensor 10 is provided for connection to a fixed point on body such as the pelvis “A”. Sensor 10 is an orientation/position sensor to transmit wirelessly (e.g. using Bluetooth) a reference point position signal to a computer 30.

A trial sensor 20 is connected on the end of a shaft 21 to a trial acetabular cup “Z”. Trial sensor 20 is an orientation/position sensor to transmit wirelessly (e.g. using Bluetooth) a trial position signal indicating the position of cup “Z” in terms of distance and angle relative to the reference point sensor.

When performing a hip replacement operation, a surgeon places the femoral head “Y” on the femur. The surgeon then places the trial cup “Z” in the pelvis and the head “Y” in the cup “Z” as shown in FIG. 1. The surgeon assesses the range of motion of the joint in flexion and extension and adjust the anteversion of the trial cup “Z” by rotating shaft 21 until the most useable range of motion is found.

A computer 30 has a receiver to receive the reference point signal from sensor 10 and the trial position signal from sensor 20, and stores the position of the cup “Z” relative to the reference point sensor 10 as a predetermined position value.

The surgeon then places acetabular cup X component in the pelvis which receives the femoral head component Y using introducer 40. The surgeon now needs to position the acetabular cup X component in the same relative position as the trial cup “Z” so it is in the optimum position.

An implant sensor 41 is connected to acetabular cup “X” through introducer 40. Implant sensor 41 is an orientation/position sensor to transmit wirelessly (e.g. using Bluetooth) an implant position signal indicating the position of cup “X” in terms of distance and angle relative to the reference point sensor 10. To do this, an algorithm may be applied to the implant position signal from sensor 41 so that the sensor 41 appears to be in the same relative position to cup “X” as the sensor 20 is relative to cup “Y”.

Computer 30 also has a receiver to receive the reference point signal from sensor 10 and the implant position signal from sensor 41, and stores the position of the cup “X” relative to the reference point sensor 10 as a comparison position value. The computer includes an algorithm which is applied to the implant position signal from sensor 41 so that the sensor 41 appears to be in the same relative position to cup “X” and the sensor 20 is relative to cup “Y”.

Computer 30 has a transmitter to transmit visual signals of the predetermined position value and comparison position value.

A pair of glasses 50 is provided for the user with one lens forming a transparent screen 51 to be seen by the user whilst simultaneously viewing a patient. The screen 51 is adapted to receive wirelessly the visual signals from the computer and to create a display to compare the predetermined position value relative to the comparison position value. The user then orientates introducer 40 until the predetermined position value substantially equals the comparison position value, whereby ensuring that the acetabular cup “X” is in the same position as the trial cup “Z”. Cup “X” can then be knocked into position using the introducer. The predetermined position value may be displayed on the screen, for example, as a first mark which needs to be aligned with a second mark representing the comparison position value. Glasses 50 can incorporate a miniature optical system that presents an image at an optimum distance wherever the user looks. The predetermined position value and comparison position value may be presented in a number of configurations depending on user preference; these can for example be presented as guided targets.

The sensors used are orientation/position sensors of the type sold by Fraunhofer or as described in US 2007/0287911. Communication between the computer and the trial sensor and implant sensor preferably uses low energy technology for wireless data transfer, such as Bluetooth. The computer may be in the form of a held device as shown, e.g. similar to an iPhone (registered trade mark) with a touch screen to select programmes etc.

Further modifications will be apparent to those skilled in the art without departing from the scope of the present invention.

1. An orthopaedic system, for providing information as to the position of a prosthesis compared to a desired position of the prosthesis, comprising:
(a) a first sensor for connection to a prosthesis and to transmit a prosthesis position signal;
(b) a second sensor for connection to a body and to transmit a reference position signal;
(c) a third sensor for connection to a trial prosthesis and to transmit a trial prosthesis signal;
(d) a computer having a receiver to receive (i) said prosthesis position signal and said reference position signal, and create therefrom a first position value, and (ii) said reference position signal and said trial prosthesis signal and create therefrom a second position value, and programmed to compare said first position value with said second position value; and
(e) an output to provide information indicating the proximity of the first position value to the second position value.

2. The system of claim 1, wherein the output varies according to the proximity of the first position value to the second position value.

3. The system of claim 1, wherein the output comprises a visual signal.

4-5. (canceled)

6. The system of claim 3, comprising a direct to eye display.

7-12. (canceled)

13. The system of claim 1, wherein the prosthesis is a hip implant, a knee implant, a shoulder implant or an elbow implant.

14. The system of claim 1, comprising:
(a) a reference point sensor (the second sensor) for connection to a fixed point on a body to transmit a reference position signal;
(b) a trial sensor (the third sensor) for connection to a trial implant and to transmit a trial position signal indicating the position of the trial implant relative to the reference point sensor;
(c) an implant sensor (the first sensor) for connection to a first implant and to transmit a first implant position signal indicating the position of the first implant relative to the reference position sensor;
(d) a computer having a receiver to (i) receive said reference point signal and trial position signal to create therefrom a predetermined position value, (ii) receive said reference point signal and first implant position signal to create therefrom a comparison position value, said computer having a transmitter to transmit output signals of the predetermined position value; and
(e) an output to compare the predetermined position value with the comparison position value.

15. (canceled)

16. A kit for use in providing information as to the position of a prosthesis compared to a desired position of the prosthesis, comprising:
(a) a prosthesis comprising a first sensor to transmit a prosthesis position signal;
(b) a second sensor for connection to a body to transmit a reference position signal;
(c) a trial prosthesis having a third sensor to transmit a trial prosthesis signal;
(d) a computer having a receiver to receive (i) said prosthesis position signal and said reference position signal and create therefrom a first position value, and (ii) said reference position signal and said trial prosthesis signal and create therefrom a second position value, and programmed to compare said first position value with said second position value; and
(e) an output to provide information indicating the proximity of the first position value to the second position value.

17-22. (canceled)

23. The kit of claim 16, wherein the prosthesis is a hip implant, a knee implant, a shoulder implant or an elbow implant and the trial prosthesis is a corresponding trial implant for performing a trial reduction to determine suitable position for the hip implant, knee implant, shoulder implant or elbow implant.

24-26. (canceled)

27. The kit of claim 16, wherein the output comprises a visual signal.

28. The kit of claim 16, comprising a direct to eye display.

29. The kit of claim 16, comprising:
(a) a reference point sensor (the second sensor) for connection to a fixed point on a body to transmit a reference position signal;
(b) a trial implant comprising a trial sensor (the third sensor) to transmit a trial position signal indicating the position of the trial implant relative to the reference point sensor;
(c) a first implant comprising an implant sensor (the first sensor) to transmit a first implant position signal indicating the position of the first implant relative to the reference position sensor;
(d) a computer having a receiver to (i) receive said reference point signal and trial position signal to create therefrom a predetermined position value, (ii) receive said reference point signal and first implant position signal to create therefrom a comparison position value, said computer having a transmitter to transmit output signals of the predetermined position value; and
(e) an output to receive said output signals and to create an output to compare the predetermined position value with the comparison position value.

30. The kit of claim 29, further comprising a screen adapted to receive said visual signals and to create a display to compare the predetermined position value relative to the comparison value, said screen being adapted to be seen by an operator whilst simultaneously viewing a patient.

31. A method of providing information as to the position of a prosthesis compared to a desired position of the prosthesis, comprising:
(a) associating a first sensor with the prosthesis, said first sensor capable of transmitting a prosthesis position signal;
(b) attaching a second sensor to a body to transmit a reference position signal;
(c) associating a third sensor with a trial prosthesis to transmit a trial prosthesis signal
(d) operating a computer having a receiver to receive (i) said prosthesis position signal and said reference position signal and create therefrom a first position value, and (ii) said reference position signal and said trial prosthesis signal and create therefrom a second position value, and programmed to compare said first position value with said second position value; and
(e) generating an output from the computer that indicates the proximity of the first position value to the second position value.

32-36. (canceled)

37. The method of claim 31, comprising moving the trial implant until it is in a desired position and then transmitting the trial prosthesis signal.

38. The method of claim 31, wherein the prosthesis is a hip implant, a knee implant, a shoulder implant or an elbow implant.

39. The method of claim 31, comprising generating an output that varies according to the proximity of the first position value to the second position value.

40. (canceled)

41. The method of claim 31, comprising generating a visual signal.

42. (canceled)

43. The method of claim 31, comprising:
(a) connecting a reference point sensor (the second sensor) to a body to transmit a reference position signal;
(b) connecting a trial sensor (the third sensor) to a trial implant, or associating a trial sensor with a trial implant, to transmit a trial position signal indicating the position of the trial implant relative to the reference point sensor;
(c) connecting an implant sensor (the first sensor) to a first implant, or associating an implant sensor with a first implant, to transmit a first implant position signal indicating the position of the first implant relative to the reference position sensor;
(d) operating a computer having a receiver to (i) receive said reference point signal and trial position signal to create therefrom a predetermined position value, (ii) receive said reference point signal and first implant position signal to create therefrom a comparison position value, said computer having a transmitter to transmit output signals of the predetermined position value; and
(e) generating an output to compare the predetermined position value with the comparison position value.

44. The method of claim 43, comprising providing the output on a screen adapted to receive said visual signals and to create a display to compare the predetermined position value relative to the comparison value, said screen being adapted to be seen by an operator whilst simultaneously viewing a patient.

45. The method of claim 43, comprising providing the output on a head mounted display.

46. (canceled)
